(19) United States
(12) Patent Application Publication
(10) Pub. No.: US 2011/0039314 A1
(43) Pub. Date: Feb. 17, 2011

(54) Composition for Catalytic Amide Production and Uses Thereof

(75) Inventors: Richard C. Holz, Chicago, IL (US); Timothy Elgren, Clinton, NY (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignees: LOYOLA UNIVERSITY OF CHICAGO, Chicago, IL (US); The Trustees of Hamilton College, Clinton, NY (US)

(21) Appl. No.: 12/856,283

(22) Filed: Aug. 13, 2010

Related U.S. Application Data
(60) Provisional application No. 61/233,946, filed on Aug. 14, 2009.

(51) Int. Cl. C12P 13/02; C12N 9/88

(52) U.S. Cl. 435/129; 435/232

(57) ABSTRACT

A catalytic composition for the enzymatic conversion of nitriles to amides is disclosed. The composition contains a polymer gel and a nitrile hydratase (NHase). Also disclosed are methods of producing an amide from a nitrile using the catalytic composition.

### CROSS REFERENCE TO RELATED APPLICATIONS

This application claims the benefit of U.S. Provisional Patent Application No. 61/233,946, filed Aug. 14, 2009, incorporated herein by reference in its entirety.

### FIELD OF THE INVENTION

The present invention relates to a catalytic composition comprising a nitrile hydratase (NHase) and a polymer gel. The catalytic composition is used in methods of preparing amides from nitriles.

### BACKGROUND OF THE INVENTION

Nitriles are extensively used in the production of a broad range of specialty chemicals and drugs including amines, amides, amidines, carboxylic acids, esters, aldehydes, ketones, and heterocyclic compounds (1-4). These compounds are used in a wide array of reactions as chemical feedstocks for the production of solvents, extractants, pharmaceuticals, drug intermediates, pesticides (e.g., dichlobenil, bromoxynil, ioxynil, and buctril), and polymers (1, 3-14).

For example, acrylonitrile and adiponitrile are used in the production of polyacrylamide and nylon-66, respectively, the latter of which is one of the most important industrial polyamides derived from petroleum feedstocks (2, 11). Nylon-66 possesses many of the properties of natural fibers (i.e., forms long chain molecules of considerable elasticity) which allow it to be spun into threads, and nylon-66 can also be molded to form cogs and gears. Nylon-66 also is widely used in clothing, carpets, and ropes. However, the harsh industrial conditions required to hydrolyze nitriles to their corresponding amides (e.g., either acid or base hydrolysis) often are incompatible with the chemically-sensitive structures of many industrially and synthetically important compounds, which decreases product yields and consequently increases production costs.

Because nitriles are synthesized by plants, fungi, bacteria, algae, insects, and sponges, several biochemical pathways exist for nitrile degradation (3, 4). Enzymes involved in nitrile degradation pathways represent chemoselective biocatalysts capable of hydrolyzing nitriles under mild reaction conditions (1, 4, 6).

Nitrile hydratases (NHase, EC 4.2.1.84) catalyze the hydrolysis of a nitrile to its corresponding amide (Scheme 1) (3). Microbial NHases have a potential as catalysts in organic chemical processes because these NHase enzymes can convert nitriles to the corresponding higher value amides in a chemo-, regio-, and/or enantio-selective manner (4). For example, Mitsubishi Rayon Co. has developed a microbial process that produces about 30,000 tons of acrylamide annually using the NHase from Rhodococcus rhodochrous J1 (14-17). This process is the first successful example of a bioconversion process for the manufacture of a commodity chemical.

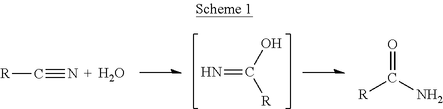

NHases are metalloenzymes that contain either a non-heme Fe(III) ion (Fe-type) or a non-corrin Co(III) ion (Co-type) in their active site (3, 4, 13, 17). Both Fe-type and Co-type NHases contain α₂β₂ heterotetramers, and each α subunit has a highly homologous amino acid sequence (CXYCSCX) that forms a metal binding site (18-21). The known X-ray crystal structures of both the Co— and Fe-type enzymes show that the M(III) (metal (III)) center is six coordinate with the remaining ligands being three cysteine residues and two amide nitrogens. Two of the active site cysteine residues are post-translationally modified to cysteine-sulfinic acid (—SO₂H) and cysteine-sulfenic acid (—SOH) yielding an unusual metal coordination geometry, which has been termed a “claw-setting” (FIG. 1). In general, it has been observed that Fe-type NHases preferentially hydrate small aliphatic nitriles, whereas Co-type NHases preferentially hydrate aromatic and halogenated aromatic nitriles (4).

A major obstacle in the use of enzymes in general, and NHases specifically, in organic synthetic processes is the difficulty in separating the enzyme from the synthetic reaction mixture (1, 4). A second obstacle is the desired use of aprotic solvents in organic synthetic reaction mixtures, which render most enzymes inactive (22, 23). One way to overcome each of these obstacles is immobilization of the enzyme within a silica glass prepared via sol-gel processing (24-26).

Encapsulated enzymes have resulted in the generation of novel functional materials that are optically transparent and sufficiently porous to permit small substrates access to the entrapped enzyme (24, 27-29). Studies have demonstrated that encapsulated proteins retain their solution structure and native function while residing in the hydrated pore of the sol-gel (24). Moreover, nanoscopic enzyme confinement within a sol-gel stabilizes the protein against thermal and proteolytic degradation (24, 30). These physical properties permit the broad application of sol-gel:protein materials as chemical sensors, separation media, and heterogeneous catalysts (31, 32). Another benefit of sol-gel encapsulation of enzymes, in general, is that such catalytic materials are readily separable from a reaction mixture by simple decanting or centrifugation.

WO 2007/086918 discloses the production of hydrogen gas using a composite material containing a polymer gel, a photocatalyst, and a protein-based H₂ catalyst, such as a hydrogenase, encapsulated in the polymer gel. The immobilization of an active hydrogenase by encapsulation in a porous polymer gel is discussed in T. E. Elgren et al., Nanoletters, Vol. 5, No. 10, pages 2085-87 (2005).

The encapsulation of horseradish peroxide in a sol-gel, and its use as a catalytic material for peroxidation, is discussed in K. Smith et al., J. Am. Chem. Soc., 124, pages 4247-4252 (2002). Nitrile hydratase is discussed in Ito et al. U.S. Pat. No. 5,807,730.

Attempts to develop enzymatic methods to produce amides on a commercial scale have been deficient. Accordingly, the present invention is directed to a composition and method for the facile conversion of nitriles to commercially significant quantities of amides in a single reaction step under mild conditions.

### SUMMARY OF THE INVENTION

The present invention is directed to catalytic compositions and methods of producing amides from nitriles, both aliphatic and aromatic, using the catalytic compositions. In one aspect, the present invention relates to a catalytic composition for amide production comprising a polymer gel and a nitrile hydratase (NHase). The nitrile hydratase can be a Co-type nitrile hydratase, for example, from Pseudonocardia thermophilic JCM3095 (PtNHase) or an Fe-type nitrile hydratase from Comamonas testoteroni Ni1 (CtNHase).

In one aspect, the NHase is encapsulated in a polymer gel. The gel can be a sol-gel, a hydrogel, or a xerogel. Sol-gels typically comprise one or more orthosilicates.

In another aspect, the present invention relates to enzymatic methods of preparing amides from nitriles, both aliphatic and aromatic, in high purity and yield.

In yet another aspect, an amide is prepared from a nitrile by a method comprising
(a) providing a compound having a nitrile moiety,
(b) providing a catalytic composition comprising
   i) a polymer gel, and
   ii) a nitrile hydratase,
(c) admixing (a) and (b) in a suitable carrier under conditions sufficient to convert the nitrile moiety to an amide moiety and provide the amide.

In certain embodiments, (a) and (b) are admixed for a sufficient time at a pH of about 6.5 to about 8 and a temperature of about 20° C. to about 60° C.

In another aspect, the method of preparing an amide from a nitrile further comprises:
(d) separating (b) from the admixture of (c), and
(e) recycling (b) into a reaction mixture to convert a nitrile to an amide.

In certain aspects of the present invention, an amide compound is provided in a yield of at least 80%. In other aspects, an amide compound is provided in an enantiomeric excess of at least 95%. In yet another aspect, the nitrile is a dinitrile, and a first nitrile moiety is converted to an amide moiety and a second nitrile moiety remains a nitrile moiety.

These and other novel aspects of the present invention will become apparent from the following detailed description of the preferred embodiments.

### BRIEF DESCRIPTION OF THE FIGURES

FIG. 1 is a structural model showing the active site of the Co-type NHase from P. thermophilic.

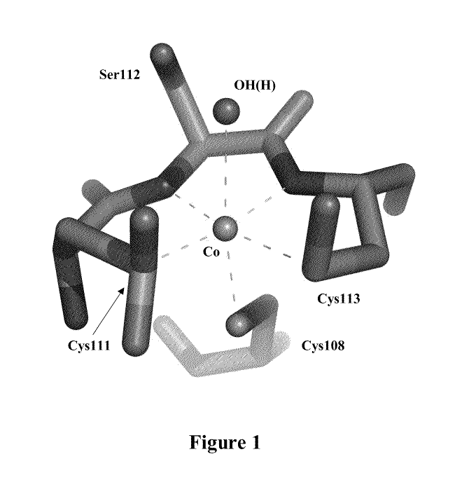

FIG. 2 contains a plot of absorbance at 242 nm vs. time (minutes) for a reaction of PtNHase:sol-gel pellets with benzonitrile in 25 mM HEPES buffer at pH 7.6 and 25° C.

FIG. 3 contains a plot of absorbance vs. wavelength (nm) for CtNHase in 100 mM HEPES buffer at pH 7.2 and 40 mM butyric acid.

FIG. 4 is an X-band EPR spectrum of CtNHase in 100 mM HEPES buffer at pH 7.2.

FIG. 5 contains a plot of absorbance at 242 nm vs. time (minutes) for a reaction of PtNHase:sol-gel pellets with benzonitrile in methanol at 25° C.

### DETAILED DESCRIPTION OF THE PREFERRED EMBODIMENTS

The present invention is directed to the enzymatic formation of an amide from a nitrile using an NHase encapsulated in a polymer gel.

Unless defined otherwise, all technical and scientific terms used herein have the same meaning as commonly understood by one of ordinary skill in the art to which this invention pertains.

Immobilization of enzymes and proteins within polymer matrices prepared by sol-gel processing has provided functional biomaterials. In many instances, these materials are optically transparent and sufficiently porous to permit small substrates access to the entrapped enzyme. As used herein, the term “porous” with respect to a present sol-gel means that sol-gel has a sufficient porosity for a nitrile of interest to pass through the surface of the sol-gel into the interior of the sol-gel for contact with an enzyme entrapped in the sol-gel.

It has been demonstrated that encapsulated proteins retain their solution structure and native function while residing in a hydrated pore within the sol-gel matrix. This nanoscopic confinement stabilizes proteins against thermal and proteolytic degradation, inhibits intermolecular disproportionation, and allows enzymatic reactions to run in aprotic solvents.

Therefore, the present invention is directed to a biomaterial that hydrolyzes nitriles to their corresponding higher value amides under mild conditions (e.g., room temperature and physiological pH). The biomaterial utilizes a Co-type nitrile hydratase and/or an Fe-type nitrile hydratase, and preferably, the thermally stable Co-type nitrile hydratase from Pseudonocardia thermophile JCM 3095 (PtNHase) and the Fe-type nitrile hydratase from Comamonas testosteroni (CtNHase).

PtNHase and CtNHase are preferred because CtNHase preferentially hydrates small aliphatic nitriles, whereas PtNHase exhibits a greater affinity for aromatic and halogenated aromatic nitriles. The range of nitriles that can be hydrolyzed therefore is extensive. Either PtNHase or CtNHase is encapsulated in a sol-gel material and the catalytic activity determined. The breadth and selectivity of the nitrile substrates that can be hydrolyzed is determined, as is the reactivity of the sol gel:enzyme biomaterials in a continuous reactor with both protic and aprotic solvent mixtures. The present NHase:sol-gel biomaterials utilize petroleum feedstock precursors for the formation of amides. The present sol-gel catalytic compositions therefore have applications in the refining of petroleum products.

Several NHase-containing bacteria have been entrapped in hydrogels, such as calcium alginate (1). However, entrapment of purified enzymes is a preferred biocatalyst for nitrile-containing compounds. In particular, complex nitriles having other hydrolyzable groups that can be degraded in side-reactions within a bacterial cell require purified NHase enzyme catalysts. In addition, processes that must avoid carboxylate formation also require purified NHase biocatalytic materials because other enzymes in the bacterial nitrile degradation pathway, such as nitrilases, convert amides to carboxylates (1). Purified enzymes also eliminate the need to have nitrile substrates pass across cell membranes of the bacteria which decreases the yield of recoverable products. Therefore, it has been found that encapsulating purified NHase enzymes in sol-gel materials provides a biocatalytic composition capable of hydrolyzing nitriles to their corresponding higher value amides under mild conditions, while avoiding the production of unwanted by-products.

The present invention therefore provides a catalytic composition comprising an NHase enzyme and a polymer gel. In particular, the catalytic composition comprises an NHase enzyme encapsulated in a sol-gel, i.e., a sol-gel:NHase. The sol-gel:NHase catalysts hydrolyze a large variety of both alkyl and aryl nitriles to their corresponding amides under mild conditions (e.g., room temperature and neutral pH). By preparing the sol-gel:NHase catalysts and determining the breadth of their reactivity, improved and/or expanded use of petroleum feed-stocks can be achieved.

In addition, the present invention provides novel catalysts that can be used in the synthesis of organic molecules for use in a wide variety of applications ranging from pharmaceuticals to specialty chemicals. The preferred nitrile hydratases are the thermally stable Co-type NHase from Pseudonocardia thermophile JCM 3095 (PtNHase) and the Fe-type NHase from Comamonas testosteroni (CtNHase). CtNHase preferentially hydrates aliphatic nitriles, whereas PtNHase preferably hydrates aromatic and halogenated aromatic nitriles. The E. coli expression systems for both PtNHase and CtNHase are known, and both enzymes have been purified to homogeneity.

In accordance with the present invention, PtNHase and CtNHase are encapsulated in sol-gel materials and their catalytic activities determined. In particular, both PtNHase and CtNHase are encapsulated in hydro- and zero-gels using tetramethyl orthosilicate (TMOS). These materials are characterized via UV-Vis and/or EPR spectroscopy, as well as SEM. The effect of temperature, pH, and ionic strength on the catalytic ability of these materials also is examined.

Enzyme encapsulation in silica-derived sol-gel materials has been demonstrated for a wide variety of enzymes, see, for example, I. Gill, Chem. Mater., 13, 3404-3421 (2001) and D. Avnir et al., J. Mater. Chem., 16, 1013-1030 (2006).

The gentle conditions typically used for encapsulating proteins follow the acid or base catalyzed condensation of SiRₙ(OH)₄₋ₙ, which leads to formation of the silica-oxo network of the gel. Alkoxysilanes, such as tetramethoxysilane (TMOS) and tetraethoxysilane (TEOS), are the typical starting materials from which hydroxy silanes are derived.

The breadth and selectivity of substrates degraded by the PtNHase and CtNHase:sol-gel materials also is investigated. In particular, the kinetic parameters of the PtNHase and CtNHase:sol-gel materials in the presence of a wide variety of alkyl and aryl nitriles is examined. A series of nitrile substrates are tested in order to assess the ability of a NHase:sol-gel catalyst to hydrolyze nitriles to amides in a chemo-, regio-, and/or enantio-selective manner.

The reactivity of the novel sol-gel:NHase biomaterials in a continuous reactor with both protic and aprotic solvents also is examined. The reaction rates of PtNHase and CtNHase:sol-gel materials in protic and aprotic solvents, as well as aprotic solvent:water mixtures, are examined in order to determine the breadth of solvents and reaction conditions that can be used in the conversion of nitriles to amides.

### Procedures and Methods

Encapsulation of PtNHase and CtNHase in sol-gel materials and determination of catalytic activity. Encapsulation of PtNHase and CtNHase is achieved by preparing sol-gels of varying composition. In preliminary experiments, hydro- and zero-gels of PtNHase, using tetramethyl orthosilicate (TMOS), are prepared using established protocols (33). In particular, a 5:1 TMOS:water (H₂O) mixture under acidic conditions is used to initiate the sol-forming condensation reaction. This solution is sonicated at 2° C. for 20 minutes. The resulting sol solution (0.25 mL) is added to a 50 to 250 μM NHase solution (0.5 mL) in 1 mM MES buffer (pH 6.5). The resulting solution is mixed briefly and cast as pellets or monoliths, which are allowed to harden for about 1 hour at 5° C. The hydrogel pellets and monoliths are washed with MES buffer solution 2-3 times and stored in buffer. Xerogels are allowed to dry and stored at 5° C. until used. CtNHase is encapsulated as both hydrogels and xerogels prepared from TMOS. PtNHase and CtNHase also are prepared as both hydro- and zero-gels of TMOS with varying amounts of tetraethyl orthosilicate (TEOS), or other alkoxide or alkyl-substituted silicates, in order to alter the hydrophobicity of the pores within the gel. The hydrophobicity of the sol-gel is systematically increased to enhance the ability to catalyze hydrolysis of more hydrophobic nitriles and help provide nitrile hydrolysis in aprotic solvents.

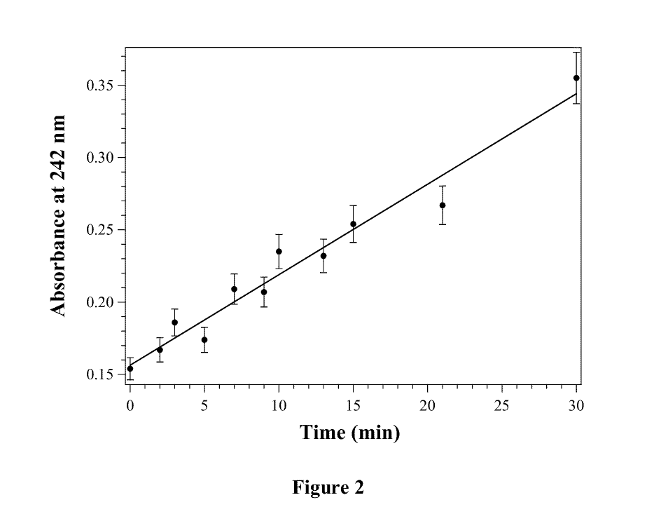

Under solution conditions, it is determined that PtNHase catalyzes the hydrolysis of benzonitrile at pH 7.6 and 25° C. with a k_{cat} value of 123 s⁻¹ and a Kₘ value of 18 μM, which are indistinguishable from previously reported values (k_{cat}=120 s⁻¹ and Kₘ=19 μM) (21). Likewise, it is found that CtNHase catalyzes the hydrolysis of cyanovaleric acid at pH 7.2 and 25° C. with a k_{cat} value of 0.23 s⁻¹ and a Kₘ value of 2,500 μM, which also are indistinguishable from previously reported values (k_{cat}=0.26 s⁻¹ and Kₘ=3,200 μM) (34). In addition, PtNHase:sol-gel pellets react readily with benzonitrile as determined by the observed increase in absorption at 242 nm (FIG. 2). Therefore, the present sol-gel:NHase catalysts display enzymatic properties, including substrate recognition, as observed for NHases in solution.

SEMs of the present sol-gel:NHase materials demonstrate the porous nature of the sol-gel surface (35), which confirms solution/substrate access to the encapsulated enzyme. Remarkably, it is found that PtNHase:sol-gel catalytic pellets can be removed from the reaction vessel, rinsed, dried, and reused weeks latter without a loss of catalytic activity. In contrast, native PtNHase and CtNHase in solution lose nearly 100% of their catalytic activity when stored under similar conditions. Therefore, sol-gel encapsulation stabilizes NHases from thermal denaturation and proteolytic cleavage to provide long lasting, robust catalysts. These data indicate that the kinetics of nitrile hydrolysis for the sol-gel:NHases is theorized to be governed by a mass transport of the nitrile substrate through the porous gel to the enzyme active site and subsequent amide product release.

To ensure that the nitrile has access to the sol-gel encapsulated NHase, as opposed to any NHase adhered to the gel surface, PtNHase:sol-gel and CtNHase:sol-gel pellets are treated with trypsin to proteolytically digest all surface accessible protein. Both PtNHase and CtNHase, in solution, are fully deactivated when treated with trypsin. The treated PtNHase:sol-gel and CtNHase:sol-gel pellets are washed copiously to remove trypsin, after which it is determined whether the pellets remain active towards benzonitrile or cyanovaleric acid, respectively. In preliminary studies, it is observed that the PtNHase:sol-gel retains detectable activity levels after treatment with trypsin, indicating that the nitrile has access to the trapped PtNHase enzyme. This trapped PtNHase enzyme is an active catalyst and is protected from trypsin digestion. It is hypothesized that, as larger nitrile substrates are used, penetration of the sol-gel material may decrease making surface bound NHases of some importance in the hydrolysis of nitriles. In solution at pH 7.6, PtNHase is stable for several hours at temperatures as high as 50° C. (21).

In preliminary studies, it also is observed that the PtNHase:sol-gel catalyst maintains activity in the hydrolysis of benzonitrile at 60° C. for over 45 minutes. These initial experiments establish that the sol-gel matrix imparts stability to the encapsulated NHase against thermal denaturation. The thermal stability of CtNHase:sol-gel encapsulated enzyme also is tested because CtNHase is not thermally stable and rapidly looses catalytic activity at temperatures above 35° C. (34).

In order to characterize the PtNHase:sol-gel and CtNHase:sol-gel catalysts and to establish that the active site metal ions remain in identical environments to that observed in solution, UV-Vis and EPR spectroscopy are used to examine and quantify the catalytic active site metal ions. This data also provides mechanistic data for the conversion of nitriles to amides via both the Co— and Fe-type NHase enzymes.

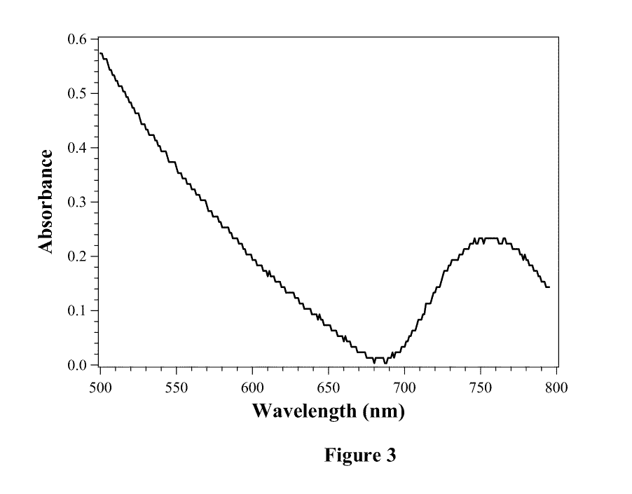

Optically transparent sol-gel glasses, suitable for UV-Vis, NMR, and EPR studies, are easily prepared using silicon, inorganic, and some hybrid sol-gels (28, 35-37). Because gels can be cast in any configuration, the ability exists to cast gels in optical cuvettes, EPR, and/or NMR tubes. UV-Vis spectra is recorded directly through the optically transparent PtNHase:sol-gel and CtNHase:sol-gel materials in optical cuvettes with a 0.5 cm path length. Based on the known molar absorptivities of the ligand-to-metal charge transfer bands at 690 (ε=1,200 M⁻¹ cm⁻¹) and 760 (ε=1,300 M⁻¹ cm⁻¹) nm for PtNHase and CtNHase (FIG. 3) respectively, the amount of encapsulated NHase enzyme can be quantified. FIG. 3 is an electronic absorption spectrum of CtNHase in 100 mM HEPES, pH 7.2 and 40 mM butyric acid.

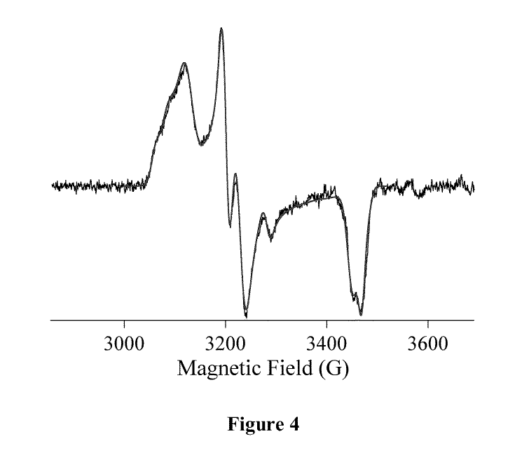

EPR spectra at X-band of the CtNHase:sol-gel material over a broad temperature range and at various powers is recorded. Xerogels shrink markedly upon drying so by casting them in NMR tubes, for example, the resulting xero-gel can be removed from the NMR tube and placed in an EPR tube. In preliminary studies, X-band EPR data on a 1 mM solution of CtNHase provided a control spectrum for comparison with sol-gel encapsulated CtNHase (FIG. 4). Integrating the observed EPR signals of both CtNHase and encapsulated CtNHase against a 2 mM Cu(II) standard quantifies the amount of NHase enzyme present in the sol-gel. FIG. 4 is an X-band EPR spectrum of CtNHase in 100 mM HEPES, pH 7.2 recorded at 10 K using 0.2 mW microwave power, 1.2 mT field modulation amplitude, 100 kHz modulation frequency, and 10.2 mT s⁻¹ sweep rate. The red traces is a simulation of the data assuming three distinct species.

The present NHase:sol-gel materials are easy-to-handle and reusable biocatalytic materials that can convert nitriles to amides under mild conditions. Another important feature of the present invention is the breadth of nitrile substrates that can be converted to amides by these encapsulated enzymatic catalysts. Therefore, the ability of PtNHase:sol-gel and CtNHase:sol-gel materials to hydrolyze a wide range of aliphatic and aromatic nitriles in a chemo-, regio-, and/or stereo-specific manner is examined (38). All of the tested substrates are commercially available or can be easily synthesized by one or two step published methods (6).

In preliminary studies, benzonitrile, which is dissolved in a 20% methanol solution in order to improve solubility, is examined. This small amount of methanol did not affect the k_{cat} values of either PtNHase or CtNHase, thus methanol is used in varying amounts as a solvent to dissolve each of the tested nitrile substrates.

The percent product formed is determined via an HPLC assay in which an aliquot of reaction mixture is removed and the reaction quenched with the HPLC mobile phase B (90% methanol, 10% water, 0.1% trifluoroacetic acid). The reaction mixture then is filtered through a 0.2 μm filter and 10 μl applied to a C₁₈ column (4.6 mm×25 cm). The initial eluting solvents are: i) mobile phase A—80% water, 20% methanol, and 0.1% trifluroroacetic acid; and ii) mobile phase B. The applied sample is resolved with a linear gradient of 0-80% mobile phase B at a flow rate of 0.5 ml/min. HPLC conditions are adjusted as needed using standard procedures known in the art to achieve separation of products from the starting material.

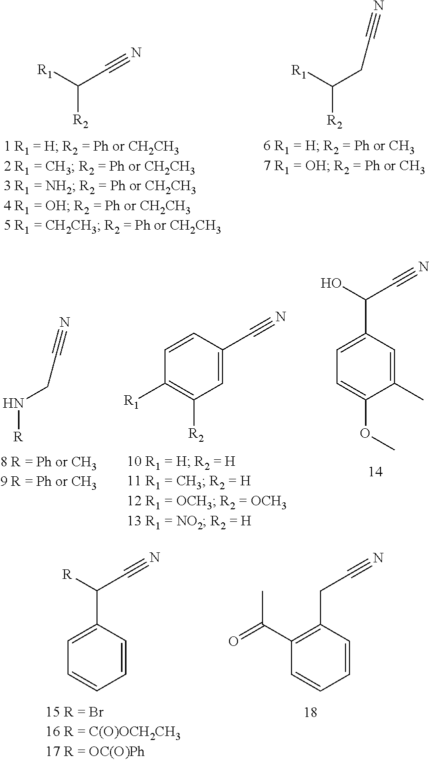

Substrate structures for conversion to an amide.

A series of aliphatic and aromatic nitriles 1-10 is examined using the soluble forms of PtNHase and CtNHase enzymes as a control because very little is known about the substrate specificity profiles of either of these enzymes, except that CtNHase preferably hydrolyzes alkyl nitriles and PtNHase preferably hydrolyzes aryl nitriles. The same substrate also is reacted with the PtNHase:sol-gel and CtNHase:sol-gel materials, and the percent product formed is compared to the percent product formed using the soluble form of the enzyme product in 30 minute reaction times at 5 minute increments. These data illustrate the breadth of nitrile substrates hydrolyzed by PtNHase:sol-gel and CtNHase:sol-gel materials, and provides information on how long the reaction must proceed to achieve ≧90% completion.

An important aspect of NHase enzymes is their ability to perform stereoselective transformations. The ability to prepare optically active compounds from nitriles has a significant impact on the synthetic methods used for high value compounds, such as pharmaceuticals, non-steroidal anti-inflammatory drugs, and agricultural chemicals. For example, the hydrolysis of (R,S)-(±)-ibuprofen nitrile by the NHase-containing bacterium Acinetobacter sp. AK226 provided (S)-(+)-ibuprofen with an enantiomeric excess (ee) of 95% (45% yield) (39).

The ability of the PtNHase:sol-gel and CtNHase:sol-gel materials to catalyze a stereoselective reaction is determined by hydrolyzing substrates such as nitrile 4 (R₂=Ph), for example. With this substrate, either the R or S enantiomer, or a racemic mixture of both, can be formed. The R and S enantiomers are kinetically resolved and physically separated using a HPLC method with a Chirobiotic T column (250×10 mm; Alltec), which allows the determination of a percent reaction of the substrate and provides an ee for the reaction. The ability of the PtNHase:sol-gel and CtNHase:sol-gel materials to hydrolyze industrially relevant molecules, such as (R,S)-(±)-ibuprofen nitrile and (±)-2-arylnitriles, also is examined.

The chemoselectivity of both the soluble forms of PtNHase and CtNHase, and the PtNHase:sol-gel and CtNHase:sol-gel materials, is investigated. A major advantage of using an NHase enzyme to catalyze the hydrolysis of nitriles is their ability to selectively react with nitriles. The chemoselectivity of PtNHase:sol-gel and CtNHase:sol-gel materials is shown by determining the percent reaction of substrates 11-18. Because classic methods of hydrolyzing nitriles involves conditions of extreme pH, which can affect other acid or alkali-labile functional groups, utilizing PtNHase:sol-gel and CtNHase:sol-gel materials to hydrolyze only nitrile moieties under neutral pH conditions without affecting other functional groups provides a major synthetic advance in the art.

Data showing that PtNHase:sol-gel and CtNHase:sol-gel materials selectively hydrolyze nitrile compounds containing ether and ester bonds (12, 14, 16, 17) indicates that these gel materials function in a chemoselective manor and also provide evidence that large bulky groups can access the encapsulated enzyme. Because the PtNHase:sol-gel materials can act as a stable catalyst at 60° C. for at least 45 minutes, a nitrile hydrolysis reaction catalyzed by the PtNHase:sol-gel material for substrates 11-18 at 60° C. in order to increase product yield also is investigated.

Another important feature of the present invention is the ability of the NHase enzymes to selectively convert only one nitrile group of a polynitrile to an amide, which has been virtually impossible using conventional methods (1, 4, 6). For example, the NHase containing bacterium R. rhodochrous K22 catalyzes the conversion of adiponitrile to cyanovaleric acid, an intermediate in the synthesis of nylon-6 (4, 40). Similarly, tranexamic acid, a homeostatic drug, was obtained by the selective hydrolysis of trans-1,4-dicyano cyclohexane by the bacterium Acremonium sp (40). In both cases, the carboxylic acid is obtained due to further intracellular reaction by a nitrilase, which converts amides to acids.

The regioselectivity of both PtNHase and CtNHase in solution, and the PtNHase:sol-gel and CtNHase:sol-gel materials, is investigated by examining dinitrile substrates 19-21. The stepwise selectivity of these catalysts also is investigated by examining dinitriles 22 and 23. Data showing that PtNHase:sol-gel and CtNHase:sol-gel materials selectively hydrolyze one nitrile group in a molecule indicates that these materials can function in a regioselective manner. The present invention therefore provides synthetic methodologies for the preparation of a wide range of molecules using dinitrile starting materials.

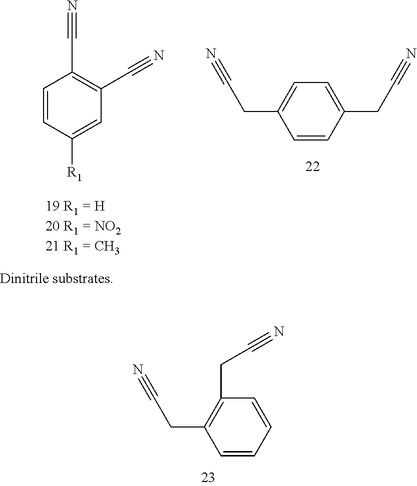

The reactivity of the present NHase:sol-gel materials in a continuous reactor with both protic and aprotic solvent mixtures is demonstrated. The remarkable stability of the NHase:sol-gel materials and the mechanistic simplicity of the hydrolysis reaction also permit a continuous synthetic method in a continuous reactor. A continuous reactor is a necessity for use of the NHase:sol-gel materials in industrial synthetic organic processes to quickly and easily hydrolyze nitriles to amides.

In order to monitor how long a present NHase:sol-gel material retains its activity for practical use in a continuous reactor, PtNHase:sol-gel or CtNHase:sol-gel catalytic pellets are positioned at the bottom of a 10 cm chromatography column and a continuous flow of fresh nitrile substrate is passed through the column. The effluent is monitored continuously using UV-Vis, HPLC, and/or LC-MS to detect hydrolysis products. A similar reactor using an encapsulated metallo aminopeptidase, namely the methionine aminopeptidase from Pyrococcus furiosus (PfMetAP-II) has been used. The pfMetAP-II:sol-gel material remains fully active after three continuous weeks of reacting at pH 7.5 at room temperature. In a separate experiment, a sol-gel encapsulated horseradish peroxidase (HRP:sol-gel) is shown to be a reusable catalyst. However, repeated use of the HRP:sol-gel resulted in diminished activity and bleaching of the chromophore associated with the active site heme presumably due to oxidative damage (28). No loss of activity was observed for the pfMetAP-II:sol-gel. The ability of the PtNHase:sol-gel and CtNHase:sol-gels to react continuously with the wide variety of nitriles and dinitriles, such as nitriles 1-23, is investigated.

In addition, the pH, temperature, and ionic strength of the substrate solution is varied in order to establish the optimum conditions for a continuous reactor for each nitrile.

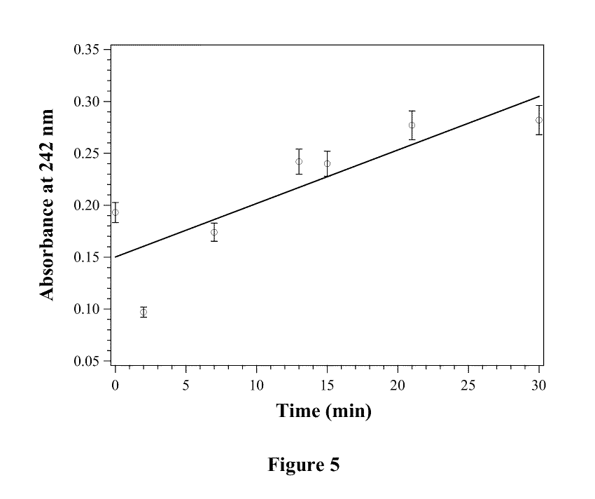

Unexpectedly, it is discovered that the PtNHase:sol-gel pellets in the xerogel state placed in methanol were able to hydrolyze benzonitrile (FIG. 5). Because only one mole of water is consumed in each catalytic cycle, it is theorized that enough water is present in the sol-gel or in the methanol to allow the encapsulated enzyme to remain catalytic.

The reaction products formed by PtNHase:sol-gel and CtNHase:sol-gel materials in organic solvents, such as methanol, are examined via HPLC and LC-MS, as is a search for potential by-products (Scheme 2) produced, for example, by methanolysis (Compound B). The ability of a present NHase:sol-gel materials to hydrolyze nitriles in other organic solvents, such as ethanol, DMSO, and THF, as well as water:organic solvent mixtures, also is investigated.

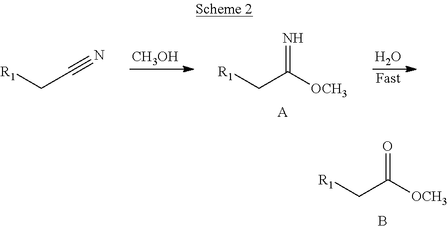

The ability to catalyze a nitrile to amide reaction in organic solvents increases the utility of NHase:sol-gel materials by increasing the number of substrates that can be hydrolyzed. The breadth of substrates, and the chemo-, regio-, and/or stereo-specific manner, that the present NHase:sol-gel materials can produce amides from nitriles in organic solvents, such as methanol is determined, using the substrates 1-23 listed above. The percent product formed as a function of time in the organic solvent is compared to the percent product obtained in buffered aqueous solutions. This data provides the reaction conditions for a wide variety of nitriles to amides which provide new avenues for the synthesis of a wide variety of industrially important petrochemicals.

The present invention therefore provides NHase materials that are organic synthetic tools that retain catalytic function. The present NHase:sol-gel materials can be cast into any desired shape, and if cast as pellets, for example, can be added in a catalytic amount to a reaction mixture and simply filtered or decanted after a prescribed reaction time. These pellets can be dried, stored for extended periods, and reused multiple times. Moreover, the present NHase:sol-gel materials are functional biomaterials capable of hydrolyzing nitriles in a chemo, regio, and stereoselective manner from a variety of nitrile substrates. Accordingly, synthetic chemists have new avenues to design synthetic methodologies using nitriles as starting materials, particularly because conversion of a nitrile moiety to the corresponding amide occurs under mild temperature and pH conditions, which helps avoid unwanted side reactions.

### Example

### Hydrolysis of Acrylonitrile

Hydrolysis of acrylonitrile was performed using PtNHase under solution conditions. PtNHase reacted readily in 500 mM acrylonitrile at pH 7.5 in 100 mM phosphate buffer to produce the corresponding amide (acrylamide). No acid byproducts were detected from the reaction, as assessed by HPLC assay performed in accordance with the methods described herein.

Hydrolysis of acrylonitrile also was performed using PtNHase:sol-gel pellets prepared as described herein. The PtNHase:sol-gel pellets reacted readily in neat acrylonitrile to produce the corresponding amide (acrylamide). No acid byproducts were detected from the reaction, as assessed by HPLC assay performed in accordance with the methods described herein. Because only one mole of water is consumed in each catalytic cycle, it is theorized that enough water is present in the sol-gel or in the acrylonitrile to allow the encapsulated enzyme to remain catalytic. Encapsulated PtNHase demonstrated increased stability compared to PtNHase enzyme in solution. While not intending to be bound by theory, encapsulation of PtNHase may improve protein stability by inhibiting protease degradation, providing protection from heat and/or chemical denaturation, and/or providing a strong hydrogen bonding network that assists the encapsulated enzyme in retaining its folded structure.

### REFERENCES

1. V. Mylerova et al., (2003), Cur. Org. Chem., 7, 1-17.
2. M. Kobayashi et al., (1992), Trends Biotechnol., 10, 402-408.
3. J. A. Kovacs, (2004), Chem Rev., 104, 825-848.
4. A. Banerjee et al., (2002), Appl Microbiol Biotechnol., 60, 33-44.
5. U. Heinemann et al., (2003), Appl Microbiol Biotechnol., 63, 274-281.
6. D. Brady et al., (2004), Appl Microbiol Biotechnol., 64, 76-85.
7. M. X. Wang et al., (2003), Org Biomol Chem., 1, 535-540.
8. M. X. Wang et al., (2003), J Org Chem., 68, 4570-4573.
9. M. X. Wang et al., (2003), J Org Chem., 68, 621-624.
10. M. X. Wang et al., (2005), J Org Chem., 70, 2439-2444.
11. R. Padmakumar et al., (1999), Appl Biochem Biotechnol., 77-79, 671-679.
12. A. Glieder et al., (2003), Angew Chem Int Ed Engl., 42, 4815-4818.
13. T. C. Harrop et al., (2004), Acc Chem Res., 37, 253-260.
14. T. Nagasawa et al., (1995) Pure Appl. Chem., 67, 1241-1256.
15. T. Nagasawa et al., (1993), Appl. Microbiol. Biotechnol., 40.
16. S. Nagashima et al., (1998), Nat Struct Biol., 5, 347-351.
17. I. Endo et al., (2001), J Inorg Biochem., 83, 247-253.
18. S. Hourai et al., (2003), Biochem Biophys Res Commun., 312, 340-345.
19. W. Huang et al., (1997), Structure, 15, 691-699.
20. A. Miyanaga et al., (2001), Biochem Biophys Res Commun., 288, 1169-1174.
21. A. Miyanaga et al., (2004), Eur J Biochem., 271, 429-438.
22. F. H. Arnold, (1988), Protein Eng., 2, 21-25.
23. K. Griebenow et al., (1996), J. Am. Chem. Soc., 118, 11695-11700.
24. I. Gill (2001), Chem. Matter, 13, 3403-3421.
25. B. C. Dave et al., (1994), Anal. Chem., 66, 1120A.
26. B. Dunn et al., (1998), Acta Mater., 46, 737.
27. L. M. Ellerby et al., (1992), Science, 255, 1113-1115.
28. K. Smith et al., (2002), J. Am. Chem. Soc., 124, 4247-4252.
29. Y. Wei et al., (2001), J Nanosci Nanotechnol., 1, 83-93.
30. D. K. Eggers et al., (2001), Protein Sci., 10, 250-261.
31. D. J. Blyth et al., (1995), Analyst, 120, 2725.
32. E. H. Lan, (1999), J. Mat. Chem., 9, 45.
33. C. G. Guizard et al., (1999), Chem. Matter, 9, 55.
34. J. M. Stevens et al., (2003), Protein Expr Purif., 29, 70-76.
35. T. E. Elgren et al., (2005), Nano Lett., 5, 2085-2087.
36. C. B. Park et al., (2002), Biotechnol Bioeng., 78, 229-235.
37. K. E. Wheeler et al., (2006), J. Am. Chem. Soc. 128, 14782 -14783.
38. L. Zhong et al., (2004), Proc Natl Acad Sci U S A., 101, 8637-8642.
39. K. Yamamoto et al., (1990), Appl. Environ. Microbiol., 56, 3125-3129.
40. H. Nishise et al., (1987), Agric. Biol. Chem., 51, 2613-2616.

What is claimed:

1. A catalytic composition comprising:
a. a polymer gel; and
b. a nitrile hydratase.

2. The composition of claim 1 wherein the nitrile hydratase is a Co-type nitrile hydratase, an Fe-type hydratase, or a mixture thereof.

3. The composition of claim 1 wherein the nitrile hydratase is PtNHase, CtNHase, or a mixture thereof.

4. The composition of claim 2 wherein the nitrile hydratase is a purified nitrile hydratase.

5. The composition of claim 1 wherein the nitrile hydratase is encapsulated in the polymer gel.

6. The composition of claim 1 wherein the polymer gel is porous.

7. The composition of claim 1 wherein the polymer gel is a sol-gel.

8. The composition of claim 7 wherein the sol-gel is a hydrogel.

9. The composition of claim 7 wherein the sol-gel is a xerogel.

10. The composition of claim 7 wherein the sol-gel comprises tetramethyl orthosilicate and optionally tetraethyl orthosilicate.

11. The composition of claim 5 in a form of a pellet.

12. A method of preparing an amide from a nitrile comprising:
(a) providing a compound having a nitrile moiety,
(b) providing a catalytic composition of claim 1,
(c) admixing (a) and (b) in a suitable carrier under conditions sufficient to convert the nitrile moiety to an amide moiety and provide the amide.

13. The method of claim 12 wherein (a) and (b) are admixed for a sufficient time at a pH of about 6.5 to about 8 and a temperature of about 20° C. to about 60° C.

14. The method of claim 12 further comprising:
(d) separating (b) from the admixture of (c); and
(e) recycling (b) into a reaction mixture to convert a nitrile to an amide.

15. The method of claim 12 wherein the catalytic composition comprises a nitrile hydratase encapsulated in a polymer gel.

16. The method of claim 12 wherein the suitable carrier comprises an aprotic solvent.

17. The method of claim 12 wherein the suitable carrier comprises water, methanol, ethanol, dimethyl sulfoxide, tetrahydrofuran, or a mixture of two or more of water, methanol, ethanol, dimethyl sulfoxide, and tetrahydrofuran.

18. The method of claim 12 wherein compound (a) is a dinitrile, and a first nitrile moiety is converted to an amide moiety and a second nitrile moiety remains a nitrile moiety.

19. The method of claim 12 wherein the amide compound is provided in a yield of at least 80%.

20. The method of claim 12 wherein the amide compound is provided in an enantiomeric excess of at least 95%.

21. The method of claim 12 wherein the nitrile comprises an aliphatic nitrile.

22. The method of claim 12 wherein the nitrile comprises an aromatic nitrile.
